(19) United States
(12) Patent Application Publication
(10) Pub. No.: US 2024/0150787 A1
(43) Pub. Date: May 9, 2024

(54) ONCOLYTIC VIROTHERAPY WITH INDUCED ANTI-TUMOR IMMUNITY

(71) Applicant: University of Houston System, Houston, TX (US)

(72) Inventors: Shaun Xiaoliu ZHANG, Houston, TX (US); Xinping FU, Houston, TX (US)

(21) Appl. No.: 17/773,277

(22) PCT Filed: Oct. 29, 2020
(86) PCT No.: PCT/US20/57820

§ 371 Date: Apr. 29, 2022

Related U.S. Application Data
(60) Provisional application No. 62/929,425, filed on Nov. 1, 2019.

(51) Int. Cl. C12N 15/86; A61K 35/763; A61P 35/00; C07K 14/195; C07K 14/485; C07K 16/32

(52) U.S. Cl. CPC C12N 15/86; A61K 35/763; A61P 35/00; C07K 14/195; C07K 14/485; C07K 16/32; C07K 2318/20; C07K 2319/02; C12N 2710/16622; C12N 2710/16632; C12N 2710/16643; C12N 2710/16662; C12N 2710/16671; C12N 2830/50

(57) ABSTRACT

Provided are improved oncolytic viruses with increased bystander cell killing and induced anti-tumor immunity. The oncolytic viruses include an oncolytic herpes virus backbone genetically modified to encode a tumor cell binding component and an immunoglobulin (Ig) binding component.

### FIELD OF THE INVENTION

This invention relates generally to methods and constructs for viral oncotherapy.

### BACKGROUND OF THE INVENTION

Without limiting the scope of the invention, its background is described in connection with existing oncolytic virotherapeutics. Oncolytic viral therapy for cancer relies on use of oncolytic viruses, which are defined by their ability to selectively replicate in and destroy tumor cells without harming normal cells.

Oncolytic viruses can kill cancer cells in many different ways, ranging from direct virus-mediated cytolysis to a variety of cytotoxic immune effector mechanisms. However, current oncolytic virotherapy is limited by sub-optimal oncolytic activities, susceptibility to suppression by innate or adaptive immune effectors, and a limited ability to induce tumor specific immune responses, particularly to neoantigens.

Thus, genetic engineering has been employed throughout the past decade to circumvent these issues and generate oncolytic viral strains with superior clinical activity. See e.g. Russell S J, et al. Oncolytic virotherapy. Nat Biotechnol 30 (2012) 658-70. In 2015, Amgen gained FDA approval for the first genetically engineered virus to treat melanoma. Pol, J. et al. Oncoimmunology 5(1) (2016) e1115641. The injectable oncolytic virus, termed talimogene laherparepvec or “T-Vec,” originated with a primary isolate of human herpes simplex virus 1 (HSV-1) known as JS1 (ECACC Accession Number 01010209) that natively demonstrated enhanced oncolytic activity. JS1 was attenuated by functionally deleting both copies of RL1 (encoding the neurovirulence factor ICP34.5), as well as US12 (encoding ICP47), and a cassette encoding human granulocyte macrophage colony-stimulating factor (GM-CSF) under the control of the cytomegalovirus immediate-early promoter was inserted into the non-functional RL1 loci. The addition of GM-CSF favors the recruitment and activation of antigen-presenting cells thus promoting the initiation of a tumor-targeting immune response.

T-Vec has shown measurable and in some cases durable therapeutic efficacy in a relatively small percentage of melanoma patients. There is no doubt that the therapeutic efficacy of this and other oncolytic viruses should and can be further improved. One improvement strategy is to potentiate the ability of virotherapy in inducing antitumor immunity. Several approaches have been reported in this attempt, mostly by incorporating immune stimulatory genes into the viral genome to enhance tumor antigen presentation for induction of T cell immunity. Provided herein is an alternative strategy.

### SUMMARY OF THE INVENTION

Improved oncolytic viruses with increased bystander cell killing and induced anti-tumor immunity are provided herein, including methods of making and using such viral constructs in the treatment of cancer.

According to one aspect of the disclosure there is provided an improved oncolytic virus comprising an oncolytic virus backbone genetically modified to encode and direct secretion from infected cells of a chimeric molecule comprising a tumor cell binding component and an immunoglobulin (Ig) binding component, wherein the secreted two chimeric molecule increases bystander cell killing and anti-tumor immunity in the presence of anti-tumor antibodies. In certain aspects the immunoglobulin binding component includes at least one Ig-binding “B” domain derived from a Peptostreptococcal Protein L. In other embodiments the immunoglobulin binding component includes at least four or five Ig-binding “B” domains derived from a Peptostreptococcal Protein L.

In certain embodiments the improved oncolytic virus is constructed on Herpes Simplex Virus Type 1 (HSV1) backbone while in other embodiments the oncolytic virus backbone is based on Herpes Simplex Virus Type 2 (HSV2). In particular embodiments, the HSV1 backbone comprises at least one deletion of ICP34.5. In other embodiments, the HSV2 backbone comprises an N-domain deletion of an ICP10 enabling selective replication in tumor cells

In certain embodiments the tumor cell binding component of the improved oncolytic virus is an affibody that binds to a tumor antigen. In other embodiments, the tumor cell binding component is ligand that binds to a tumor antigen in the form of a cell surface receptor expressed or over expressed on tumor cells. In still other embodiments, the tumor cell binding components are single chain antibodies (scFvs) or single domain antibodies (nanobodies). In certain embodiments the tumor antigen is selected from human epidermal growth factor receptor 2 (HER2), epidermal growth factor receptor (EGFR), Erb-B2 Receptor Tyrosine Kinase 3 (ErbB3), epithelial cell adhesion molecule (EpCAM), mesothelin (MSLN), MET protooncogene, insulin-like growth factor 1 receptor (IGF1R), ephrin receptor A3 (EphA3), TNF receptor apoptosis-inducing ligand receptor 1 (TRAIL-R1), TNF receptor apoptosis-inducing ligand receptor 2 (TRAIL-R2), vascular endothelial growth factor receptor (VEGFR), receptor activator of nuclear factor κβ ligand (RANKL), programmed death-ligand 1 (PD-L1), phosphatase of regenerating liver 3 (PRL-3), human melanoma antigens recognized by T cells, and Carcinoembryonic Antigen (CEA) gene family products. In certain embodiments the human melanoma antigens recognized by T cells are selected from: Melanoma-associated Antigen 1 (MAGE-A1); Melanoma-associated Antigen A3 (MAGE-A3); B Melanoma Antigen (BAGE); G Antigen 2C (GAGE), Melanoma Antigen Recognized by T Cells 1 (MLANA), and premelanosome Protein (PMEL).

In a particular embodiment, the tumor cell binding component is a ligand that binds to a tumor antigen, for example, an extracellular domain of epidermal growth factor (EGF) that binds to an EGF receptor (EGFR) on tumor cells.

According to a further aspect, there is provided an improved oncolytic virus with increased bystander cell killing and induced anti-tumor immunity, the virus comprising an oncolytic herpes virus backbone genetically modified to encode an Affibody-Protein L (PL) cassette comprising an Anti-HER2 Affibody and a plurality of Protein L immunoglobulin binding domains fused together in frame to form an Affibody-PL that is engineered for extracellular secretion by cells infected with the oncolytic virus. In certain embodiments the Affibody-PL cassette comprises a synthetic signal peptide (Sp), the anti-HER2 Affibody (Affibody), a linker (such as (GGGS)3, (Gly)8, (Gly)6, (EAAAK)3), the plurality of Protein L immunoglobulin binding domain (1-5, with 5 being optimal), and a growth hormone polyadenylation signal (polyA).

According to another aspect, there is provided an improved oncolytic virus with increased bystander cell killing and induced anti-tumor immunity, the virus comprising an oncolytic herpes virus backbone genetically modified to encode an extracellular domain of epidermal growth factor (EGF) that binds to an EGF receptor (EGFR) on tumor cells and a plurality of Protein L domains fused together in frame to form an EGF-PL that is engineered for extracellular secretion by cells infected with the oncolytic virus.

An improved oncolytic virus with increased bystander cell killing and induced anti-tumor immunity, the virus comprising an oncolytic herpes virus backbone genetically modified to encode an extracellular domain of epidermal growth factor (EGF) that binds to an EGF receptor (EGFR) on tumor cells and a plurality of Protein L domains fused together in frame to form an EGF-PL that is engineered for extracellular secretion by cells infected with the oncolytic virus.

Also provided is a method of treating cancer comprising administering a therapeutically effective amount of the improved oncolytic viruses disclosed herein and a diluent or carrier.

### BRIEF DESCRIPTION OF THE DRAWINGS

For a more complete understanding of the present invention, including features and advantages, reference is now made to the detailed description of the invention along with the accompanying figures:

FIG. 1A and FIG. 1B depict the design of an Affibody-PL and its in vivo action mechanism in tumor microenvironment. FIG. 1A depicts the gene cassette of the chimeric molecule Affibody-PL. FIG. 1B depicts a predicted action mechanism of Affibody-PL after delivered to HER2-expressing or EGFR-expressing tumors by an oncolytic virus.

FIG. 2A provides a cartoon of the step-wise construction of FusOn-H2. FIG. 2B provides a cartoon of the step-wise construction of FusOn-PL. FIG. 2C shows an alignment of the 5 domains of Finegoldia magna Protein L as shown by Kastern. (Kastern W et al. “Structure of Peptostreptococcal Protein L and Identification of a Repeated Immunoglobulin Light Chain-binding Domain” JBC 267 (18) (1992) 12820-12825).

FIG. 3 shows the results of Affibody-PL efficiently binding to HER2 expressed on tumor cell surface. Tumor cells expressing different levels of HER2 (high on Skov3 cells, medium on MCF7 cells and completely negative on MDA-MB-231 cells) were sequentially incubated with 1) Affibody-PL supernatant, 2) anti-HA antibody of mouse origin and 3) FITC-conjugated goat anti-mouse IgG. The cells were then subject to flow cytometry analysis.

FIG. 4A and FIG. 4B show the capability of Affibody-PL in guiding PBMCs to kill HER2-expressing tumor cells. PBMCs were prepared from normal human blood and were mixed with SKOV3 tumor cells at the ratio of either 10 (R10) or 20 (R20) in the presence of 5 μg/ml of IgG and either of: medium, supernatant harvested from HEK293 cells transfected with a control vector (Ctl-Vec), or supernatant harvested from HEK293 cells transfected with Affibody-PL (Affibody-PL). Twenty-four hours later, PBMCs and the dead cells floating in the medium were removed and the remaining living cells were stained with 0.1% crystal violet-ethanol solution. FIG. 4A shows representative micrographs from each well of the three different preparations. FIG. 4B shows quantification of tumor cell killing. The stained tumor cells were lysed with 2% SDS and the released dye was measured at 595 nM wavelength using Spectramax 5 plate reader. The percentage of tumor cell killing was calculated by dividing the reading of cells in the well without adding PBMCs (and others) with the readings from each of the three wells. ★p<0.05 as compared with medium and Ctl-Vec.

FIG. 5A shows Western blot detection of Affibody-PL expressed from FusOn-Affibody-PL. Supernatants were collected from Vero cells infected with either FusOn-H2 or FusOn-Affibody-PL. After filtration through 0.1 μM filter, the supernatants were loaded for gel electrophoresis and western blotting with rabbit anti-HA tag IgG as the first antibody. FIG. 5B shows flow cytometry analysis on the binding of Affibody-PL produced from FusOn-Affibody-PL to murine tumor cells expressing HER2. The experiment was similarly conducted as in FIG. 3, with the exception that a murine colon cancer cell line that was stably transduced with HER2 (CT26-HER2) was used. FIG. 5C shows measurement on the capability of Affibody-PL in guiding PBMCs to kill HER2-expressing tumor cells. The experiment was similarly conducted as in FIG. 4B, with the exception that: 1) the cells are of murine origin (CT26-HER2 and splenocytes harvested from Balb/c mice), 2) mouse Igs were used, and 3) the supernatants were obtained from FusOn-H2 or FusOn-Affibody-PL infected cells. ★p<0.05 as compared with medium and FusOn-H2 supernatant.

FIG. 6A shows NK cell infiltration during virotherapy of FusOn-H2 and FusOn-Affibody-PL. Tumor tissues were collected 3 days after mice were treated with 1×10⁷ pfu of either PBS, FusOn-H2 or FusOn-Affibody-PL. The collected tumor tissues were divided into halves. One half was paraffin-embedded and the tissue sections were used for immunohistochemical staining for NK cells. The positively stained NK cells were indicated with black arrows. The other half was used to prepare cryosections, which were then immunohistochemically stained for both NK marker (NCR1) and ki67 (FIG. 6B). The positively stained NK cells were indicated with white arrowheads and cells positively stained for ki67 were indicated with white arrows.

FIG. 7A and FIG. 7B show the results of a therapeutic evaluation and comparison of virotherapy between FusOn-H2 and FusOn-PL. CT26-HER2 tumor cells were implanted subcutaneously. When tumors reached the approximate size of 5 mm in diameter, mice were treated intratumorally with 1×10⁷ pfu of either FusOn-PL or 1×10⁷ FusOn-H2, or PBS as a negative control. FIG. 7A shows the change of tumor size following treatment. FIG. 7B shows the results of an IFN-γ ELISPO assay on Class I neoantigen-specific antitumor immunity during virotherapy. The photos show a typical area of the wells from ELISPOT assay with the indicated neoantigen peptide or the no peptide control. ★p<0.05 as compared with either FusOn-H2 or PBS.

FIG. 8A shows the results of a therapeutic evaluation of a short term virotherapy with FusOn-H2 and FusOn-PL. The experimental procedure was identical to those described in FIG. 7A. FIG. 8A show the results of tumor size measured consecutively for three weeks before all mice were euthanized. Tumor growth ratio was calculated by dividing the tumor volume measured at the indicated time with the tumor volume immediately before the start of treatment. FIG. 8B shows the enumeration data of ELISPOT assay on the neoantigen peptides shown in Table 2 and the mixture of them. The controls include no peptide or an unrelated peptide (the ovalbumin Class II peptide (OVA 323-339). ★p<0.05 as compared with FusOn-H2. +p<0.05 as compared with PBS.

FIG. 9A shows a schematic of Synco-4 construction beginning with Synco-2D. FIG. 9B shows identification Synco-47G transformants by green fluorescence.

FIG. 10 shows expression and secretion of EGF-PL by Synco-4 in 293 cells that were either transfected with a plasmid (pCR-ul47-EGFPL) that contains the EGF-PL gene cassette or infected with Synco-4.

FIG. 11A and FIG. 11B show that EGF-PL produced by Synco-4 binds to EGFR expressed on tumor cells with high efficiency and specificity. FIG. 11A shows flow cytometry of the staining for EGFR expression on CT-26-EGFR cells but not on other tumor cells. FIG. 11B shows flow cytometry evidencing that EGF-PL in the supernatant of Synco-4 infection can strongly bind to CD26-EGFR cells.

FIGS. 12A-12G show cytotoxicity effect of Synco-4 on various tumor cell lines. Tumor cells are treated with Synco-4 at 0.003-3 MOI (Multiply of infection) and detected viability using MTT assay kit after 48 hr incubation. The data shows that Synco-4 induces dose-dependent inhibition of tumor cell proliferation on Syrian Hamster adenocarcinoma cells (HaP-T1) (FIG. 12A), Murine breast cancer cells (4T-1) (FIG. 12B), Murine colon cancer cells (CT-26) (FIG. 12C), Human glioblastoma cells (U87) (FIG. 12D), Human pharynx squamous carcinoma cells (FaDu) (FIG. 12E), Human hepatocellular carcinoma cell (HEPG2) (FIG. 12F), and Human osteosarcoma cells (MNNG-HOS1) (FIG. 12G).

FIG. 13 depicts results showing that Synco-4 has an enhanced antitumor activity over the parental synco-2D against CD26-EGFR tumor.

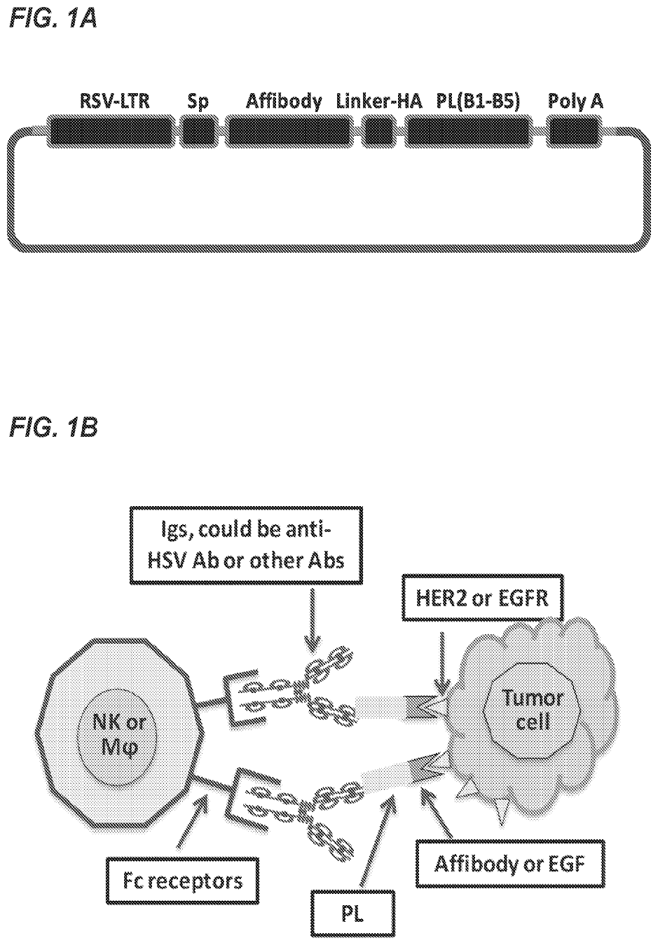

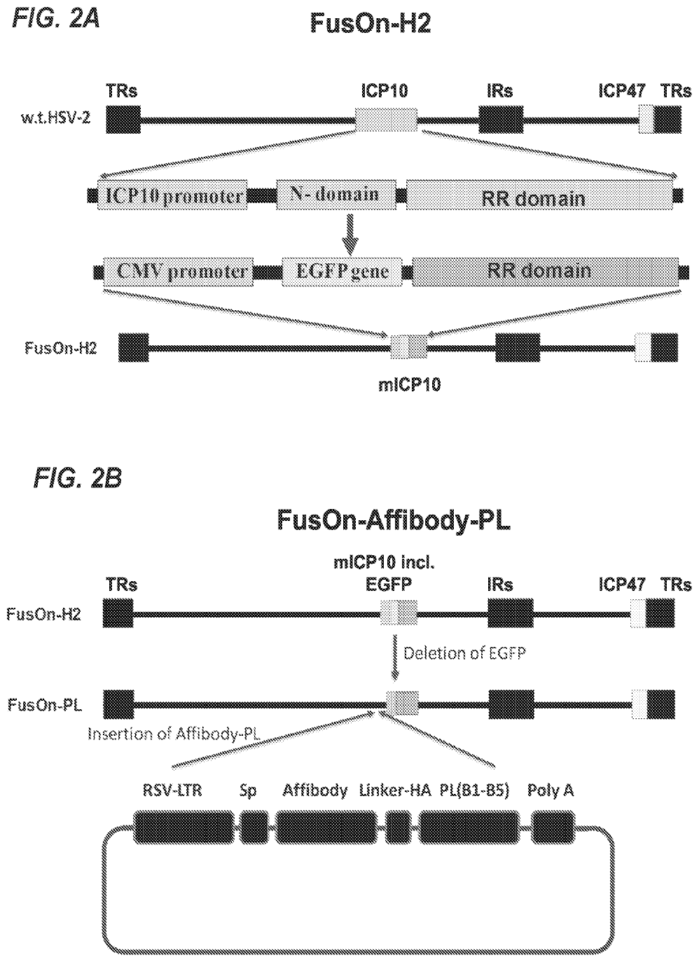

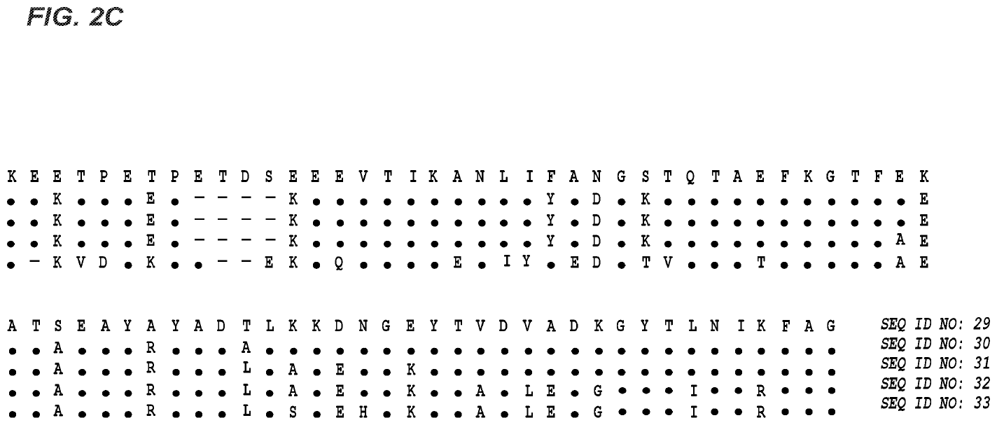

SUMMARY OF THE SEQUENCE LISTING

| SEQ ID NO: | Description |
|---|---|
| SEQ ID NO: 1 | Codon optimized sequence encoding Finegoldia magna |
| | Protein L 1^{st} kappa light chain binding domain |
| SEQ ID NO: 2 | Codon optimized sequence encoding Finegoldia magna |
| | Protein L 5 kappa light chain binding domains |
| SEQ ID NO: 3 | Anti-HER2 Affibody |
| SEQ ID NO: 4 | Rous sarcoma virus (RSV) long terminal repeat |
| | (LTR) of Affibody-PL (FIG. 1A) |
| SEQ ID NO: 5 | signal peptide (Sp) (synthetic) of FIG. 1A |
| SEQ ID NO: 6 | Linker-HA tag (HA) of FIG. 1A |
| SEQ ID NO: 7 | polyadenylation signal (polyA) (bovine growth |
| | hormone) of FIG. 1A |
| SEQ ID NO: 8 | Modified ICP10 domain (mICP10) of FIG. 2A |
| | including CMV promoter and EGFP |
| SEQ ID NO: 9 | Affibody-PL gene cassette |
| SEQ ID NO: 10 | EGF ligand |
| SEQ ID NO: 11 | HSV-2 ICP10 left flanking region |
| | amplification primer |
| SEQ ID NO: 12 | HSV-2 ICP10 left flanking region |
| | amplification primer |
| SEQ ID NO: 13 | RR domain and the right-flank region |
| | amplification primer |
| SEQ ID NO: 14 | RR domain and the right-flank region |
| | amplification primer |
| SEQ ID NO: 15 | CMV promoter-EGFP PCR amplification primer |
| SEQ ID NO: 16 | CMV promoter-EGFP PCR amplification primer |
| SEQ ID NO: 17 | entire sequence of FusOn-Affibody-PL |
| SEQ ID NO: 18 | CT26-M19 MHC class I neoantigen peptide |
| SEQ ID NO: 19 | CT26-M39 MHC class I neoantigen peptide |
| SEQ ID NO: 20 | CT26-M26 MHC class I neoantigen peptide |
| SEQ ID NO: 21 | CT26-ME1 MHC class II neoantigen peptide |
| SEQ ID NO: 22 | CT26-ME2 MHC class II neoantigen peptide |
| SEQ ID NO: 23 | CT26-ME20 MHC class II neoantigen peptide |
| SEQ ID NO: 24 | CT26-ME37 MHC class II neoantigen peptide |
| SEQ ID NO: 25 | entire sequence of Synco-4 |
| SEQ ID NO: 26 | Lox P1 |
| SEQ ID NO: 27 | LoxP2 |
| SEQ ID NO: 28 | SV40 Poly A |
| SEQ ID NO: 29 | Amino acid sequence of Finegoldia magna |
| | Protein L 1^{st} kappa binding domain |
| SEQ ID NO: 30 | Amino acid sequence of Finegoldia magna |
| | Protein L 2^{nd} kappa binding domain |
| SEQ ID NO: 31 | Amino acid sequence of Finegoldia magna |
| | Protein L 3^{rd} kappa binding domain |
| SEQ ID NO: 32 | Amino acid sequence of Finegoldia magna |
| | Protein L 4^{th} kappa binding domain |
| SEQ ID NO: 33 | Amino acid sequence of Finegoldia magna |
| | Protein L 5^{th} kappa binding domain |
| SEQ ID NO: 34 | Amino acid sequence of 5 kappa binding |
| | domain Protein L of Finegoldia magna |
| SEQ ID NO: 35 | entire sequence of Synco-2D |
| SEQ ID NO: 36 | entire sequence of FusOn-H2 |

### DETAILED DESCRIPTION OF THE INVENTION

One of the major host defense mechanisms against viral infection is via innate immune cells, which include NK cells and macrophages. They can rapidly clear the introduced oncolytic virus and thus present a major obstacle for cancer virotherapy. Provided herein are constructs and methods that redirect the infiltrating innate immune cells to attack tumor cells instead. In exemplary embodiments disclosed herein, herpes simplex virus (HSV)-based oncolytic viruses, FsOn-H2 (SEQ ID NO: 36) or Synco-2D (SEQ ID NO: 35), are armed with a secreted form of chimeric molecule that contains two separate components. In certain embodiments, component one is either an affibody (approx. 58 aa peptide) that binds with a tumor antigen such as (for example) HER2 or the extracellular domain of the epidermal growth factor (EGF) that binds to the EGF receptor (EGFR) on tumor cells. The second component is Protein L (PL) that can bind to a variety of immunoglobulins (Igs), including antibodies against HSV. In vitro experiments disclosed herein demonstrate that the secreted chimeric molecule can actively engage macrophages and NK cells with HER2 or EGFR-expressing tumor cells by simultaneously binding to the Fc receptors on the innate immune cells (through the engaged Igs) and HER2 or EGFR on tumor cells, leading to efficient killing of the latter. Subsequent evaluation in murine tumor models with limited permissiveness to FusOn-H2 or Synco-2D shows that arming the viruses with this chimeric molecule can significantly boost the therapeutic activity. Moreover, the data provided herein indicate that the combined killing effect from the engaged innate immune cells and the oncolytic virus provides an impetus to more efficiently stimulate the host's antitumor immunity than the FusOn-H2 or Synco-2D virotherapy alone. Together, the data provided herein show that arming an oncolytic virus with this strategy represents a viable way of potentiating the oncolytic and immunotherapeutic effect of a virotherapy.

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

ABBREVIATIONS: The following abbreviations are used throughout this application:

| | |
|---|---|
| ADCC | antibody-dependent cell-mediated cytotoxicity |
| BAGE | B Melanoma Antigen (OMIM 605167) |
| CEA | Carcinoembryonic Antigen gene family, |
| | including OMIM 114890 |
| EGFR | Epidermal Growth Factor Receptor, aka |
| | ErbB-1, HER1 in humans (OMIM 131550) |
| ErbB3 | Erb-B2 Receptor Tyrosine Kinase 3 (OMIM190151) |
| EphA3 | ephrin receptor A3 (OMIM 179611) |
| GAGE | G Antigen 2C (OMIM entry 300595) |
| gp75 | intracellular melanosomal membrane glycoprotein |
| | (OMIM115501) |
| HER-2 | Human Epidermal Growth Factor Receptor 2 or |
| | HER2/neu, aka Receptor tyrosine-protein kinase |
| | erbB-2, also known as CD340, proto-oncogene |
| | Neu, Erbb2 (rodent), or ERBB2 (human) (OMIM164870) |
| HSV | Herpes Simplex Virus |
| IGF1R | insulin-like growth factor 1 receptor (OMIM147370) |
| MAGE-1 | Melanoma-associated Antigen 1 (aka MAGEA1, OMIM 300016) |
| MAGE-3 | Melanoma antigen, Family A3 (OMIM 300174) |
| MLANA | Melanoma Antigen Recognized by T Cells 1, aka MART-1, |
| | MelanA (OMIM 605513) |
| MSLN | Mesothelin (OMIM601051) |
| MET | MET Protooncogene (aka Hepatocyte growth factor receptor, |
| | OMIM 164860) |
| NK | Natural Killer |
| PL | Protein L - immunoglobulin (Ig) binding protein derived from a |
| | cell wall protein generated by certain strains of the gram-positive |
| | anaerobic bacteria Peptostreptococcus magnus |
| | (now known as Finegoldia magna). |
| PMEL | Premelanosome Protein (aka melanocyte protein 17, PMEL17, |
| | PMELGP100, gp100, OMIM 155550) |
| RANKL | receptor activator of nuclear factor κβ ligand (OMIM602642) |
| PD-L1 | programmed death-ligand 1 (aka CD274, OMIM605402) |
| PTP4A3 | Protein-Tyrosine Phosphatase, Type 4A, 3 (aka phosphatase of |
| | regenerating liver 3, PRL-3 OMIM 606449) |
| TRAIL-R1 | TNF receptor apoptosis-inducing ligand |
| | receptor 1 (OMIM 603611) |
| TRAIL-R2 | TNF receptor apoptosis-inducing ligand |
| | receptor 2 (OMIM 603612) |
| VEGFR | vascular endothelial growth factor |
| | receptor (OMIM191306) |

To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within 5%, within 1%, and in certain aspects within 0.5%.

In order for an oncolytic virus to efficiently infect and lyse tumor cells, it has to overcome the host's defense mechanisms that are normally initiated when infectious agents are encountered. The innate immune system is the first line of such a defense response, which can be launched almost instantly as soon as the oncolytic virus is administered. As such, the innate immune response presents as a significant barrier to cancer virotherapy. The major components of innate antiviral immunity include natural killer (NK) cells, macrophages and interferons (IFNs). Studies by Fulci and others have shown that depletion of macrophages during virotherapy can significantly improve the therapeutic activity from an oncolytic herpes simplex virus (HSV). See Fulci G, et al. Cyclophosphamide enhances glioma virotherapy by inhibiting innate immune responses. Proc Natl Acad Sci USA 103(34) (2006) 12873-8. Others have shown that NK cells are recruited by oncolytic HSV to the tumor site within hours after virus administration, leading to quick clearance of the introduced virus and hence a diminished therapeutic effect in a murine glioblastoma model. These and other similar reports have clearly underscored the importance in limiting the innate antiviral immunity during cancer virotherapy.

On the other hand, the two major cellular components of innate antiviral immunity, NK cells and macrophages, have the potential capacity to kill malignant cells if properly activated or guided. It is thus plausible that a strategy may be developed to divert the infiltrating innate immune cells away from clearing the oncolytic virus during virotherapy, and to guide them to attack tumor cells instead.

Antibodies to the oncolytic virus represent another major limiting factor to the full therapeutic effect of virotherapy, as they can neutralize the oncolytic virus during its spread within tumor tissues. Thus, one approach is to exploit the massive infiltration of these innate immune cells during virotherapy by converting them to tumor-targeted effector cells. One of the important activation mechanisms for NK and macrophage is antibody-dependent cell-mediated cytotoxicity (ADCC). ADCC is triggered by the binding of the Fc portion of IgG to Fc gamma receptors (FcγRs), which becomes exposed when multiple IgG molecules are in an aggregated multimeric form (e.g., within an immune complex).

A potentially useful immunoglobulin aggregator is Protein L (“PL”), which is an immunoglobulin (Ig) binding protein derived from a cell wall protein generated by certain strains of the gram-positive anaerobic bacteria Peptostreptococcus magnus (now known as Finegoldia magna). The PL gene product is a 76 to 106-kDa protein containing four or five highly homologous, consecutive extracellular Ig-binding domains “B” domains, each 72 to 76 amino acid residues long. See Kastern W et al. “Structure of Peptostreptococcal Protein L and Identification of a Repeated Immunoglobulin Light Chain-binding Domain” JBC 267 (18) (1992) 12820-12825. The amino acid sequence of the first kappa light chain binding domain of Finegoldia magna Protein L is set out in SEQ ID NO:29. The amino acid sequence of the second kappa light chain binding domain of Finegoldia magna Protein L is set out in SEQ ID NO:30. The amino acid sequence of the third kappa light chain binding domain of Finegoldia magna Protein L is set out in SEQ ID NO:31. The amino acid sequence of the fourth kappa light chain binding domain of Finegoldia magna Protein L is set out in SEQ ID NO:32. The amino acid sequence of the fifth kappa light chain binding domain of Finegoldia magna Protein L is set out in SEQ ID NO:33. An exemplary codon optimized sequence encoding the first kappa light chain binding domain of Finegoldia magna Protein L is set out in SEQ ID NO:1. An alignment of the 5 domains of Finegoldia magna Protein L as shown by Kastern (supra) is shown in FIG. 2C. In certain embodiments, the immunoglobulin aggregator is formed by a plurality of kappa light chain binding domains of Finegoldia magna Protein L wherein each of the individual kappa light chin binding domains has at least 80% amino acid identity with the first kappa light chain binding domain of Finegoldia magna Protein L as set out in SEQ ID NO:29. In other embodiments, the immunoglobulin aggregator can be a plurality of any combination of the kappa light chain binding domains of Finegoldia magna Protein L as set out in any of SEQ ID NO:29, SEQ ID NO:30, SEQ ID NO:31, SEQ ID NO:32, and SEQ ID NO:33.

Unlike Proteins A and G that bind to the Fc region of Ig, Protein L binds to the variable regions of kappa light chains of the entire classes of Igs, including IgG, IgM, IgA, IgE and IgD. This allows the Fc region of the bound antibodies to bind to the FcγRs on innate immune cells to initiate ADCC. Most importantly, similar to Proteins A and G, Protein L also possesses multiple copies (up to five) of Ig-binding domains. This allows a single Protein L molecule to bind multiple units of Ig simultaneously, creating an aggregated multimeric form of Ig with the potential to induce Fc receptor oligomerization and hence the consequential initiation of ADCC. The amino acid sequence of the Finegoldia magna Protein L including the five kappa light chain binding domains is set out in SEQ ID NO: 34. An exemplary codon optimized sequence encoding the Finegoldia magna Protein L including the five kappa light chain binding domains is set out in SEQ ID NO:1.

In certain embodiments the immunoglobulin aggregator includes a plurality of kappa light chain binding domains each having kappa light chin binding activity and having at least 80 percent amino acid sequence homology to the amino acid sequence of the first kappa light chain binding domain of Finegoldia magna Protein L set out in SEQ ID NO:29.

The strategy of using Protein L to engage both Igs and innate immune cells to guide them to attack tumor cells can theoretically divert these two immune components away from clearing oncolytic virus, thus allowing it to fully replicate and spread within tumor tissues. As such, this strategy not only provides additional killing effect, but also potentiates the oncolytic effect of virotherapy per se.

In one embodiment provided herein, the above-mentioned features of Protein L Ig binding domains are exploited to enhance the therapeutic effect of an oncolytic virus. In this embodiment, oncolytic viruses are designed to redirect the infiltrating innate immune cells to attack tumor cells instead of clearing the introduced oncolytic virus. Specifically, a HSV-2-based oncolytic virus termed “FusOn-H2” was constructed to express a soluble form of a chimeric molecule consisting of a HER2-specific Affibody attached to Protein L. In other embodiments the HSV-1-based oncolytic virus termed “Synco-2D” was instead utilized, and the PL is linked to EGF so that the EGF-PL can target tumor cells overexpressing EGFR. The concept is applicable to other oncolytic virus constructs.

The Anti-HER2 Affibody is a short polypeptide with a strong binding affinity to the HER2 tumor associated antigen. In one embodiment the anti-HER2 Affibody has the sequence set out in SEQ ID NO: 3. In vitro experiments described herein demonstrated that the chimeric molecule can actively engage macrophages and NK cells with HER2-expressing tumor cells in the presence of Ig, leading to efficient killing of the latter. Subsequent evaluation in a murine tumor model with limited permissiveness to FusOn-H2 showed that arming the virus with this chimeric molecule significantly boosts the therapeutic activity. Moreover, data presented herein indicate that the combined killing effect from the engaged innate immune cells and the oncolytic virus provides an impetus to more efficiently stimulate the host's antitumor immunity than the FusOn-H2 virotherapy alone. Together, the data suggest that arming an oncolytic virus with this strategy represents a viable way of potentiating the oncolytic and immunotherapeutic effect of a virotherapy.

The sole oncolytic virus that has been approved by the US Food and Drug Administration (FDA) for clinical application, Imlygic or T-VEC, has shown measurable and in some cases durable therapeutic efficacy in a relatively small percentage of melanoma patients. There is no doubt that the therapeutic efficacy of this and other oncolytic viruses should and can be further improved. One improvement strategy is to potentiate the ability of virotherapy in inducing antitumor immunity. Several approaches have been reported in this attempt, mostly by incorporating immune stimulatory genes into the viral genome to enhance tumor antigen presentation for induction of T cell immunity.

Provided herein is an alternative strategy to those previously undertaken, which is designed to arm an oncolytic virus with a chimeric molecule that can guide the infiltrating innate immune cells to destroy tumor cells. This is in contrast to other strategies that are mainly focused on the host's adaptive immune cells such as T cells. The chimeric element includes a tumor cell specific targeting moiety and Protein L as an Ig binding domain. One of the unique advantages of this strategy is that it exploits the extensive infiltration of these innate immune cells during virotherapy, as lack of sufficient T cell presence in solid tumors has been considered as a major hurdle for cancer immunotherapies such as check-point blockers and other strategies designed to potentiate T effector cells. Indeed, immunohistochemistry staining on tumor tissues treated with FusOn-Affibody-PL revealed an extensive infiltration of NK cells. The combined effect of Affibody-PL in guiding the infiltration of innate immune cells and the local release of Affibody-PL or EGF-PL, together resulting in destruction of additional tumor cells, apparently contributes to additional and hence enhanced antitumor activities seen from the in vivo studies. Indeed, half of the tumor-bearing mice treated with FusOn-Affibody-PL were completely tumor-free by the end of the experiment. Subsequent challenge of these mice with fresh tumor cells failed to initiate tumor growth in these mice, indicating the possibility that a robust T-cell mediated antitumoral immunity was subsequently generated by the applied virotherapy in these mice.

Tumor antigen present on tumor cells are targetable by viral encoded molecules that recognize and bind to the tumor antigens. Examples of tumor antigens include human epidermal growth factor receptor 2 (aka HER-2 and ErbB2, currently targeted in antibody mediated therapy with trastuzumab (aka HERCEPTIN®)), epidermal growth factor receptor (EGFR; also known as ErbB1), Erb-B2 Receptor Tyrosine Kinase 3 (ErbB3), mesothelin, MET tyrosine kinase receptor, insulin-like growth factor 1 receptor (IGF1R), ephrin receptor A3 (EphA3), TNF receptor apoptosis-inducing ligand receptor 1 (TRAIL-R1), TNF receptor apoptosis-inducing ligand receptor 2 (TRAIL-R2), vascular endothelial growth factor receptor (VEGFR), the receptor activator of nuclear factor κβ ligand (RANKL) and programmed death-ligand 1 (PD-L1). New tumor antigens include the cancer-related phosphatase PRL-3, the human melanoma antigens recognized by T cells including the MAGE-1 (Melanoma-associated Antigen 1, aka MAGEA1, OMIM entry 300016), MAGE-3 (aka Melanoma antigen, Family A3, OMIM entry 300174), BAGE (aka B Melanoma Antigen, OMIM entry 605167), and GAGE families (aka G Antigen 2C, OMIM entry 300595), as well as MelanA (Melanoma Antigen Recognized by T Cells 1, aka MLANA, MART-1, OMIM entry 605513), and Premelanosome Protein (PMEL, aka melanocyte protein 17, PMEL17, PMELGP100, gp100, OMIM entry 155550). Further targets include certain gene products of the Carcinoembryonic Antigen (CEA) gene family including OMIM entry 114890).

Thus, the targeting element of the chimeric molecule expressed by the virus specifically binds a tumor antigen such as those identified above. In certain embodiments, the targeting element expressed by the virus is an affibody, while in other embodiments the targeting element is single-chain variable fragment (scFv) or single domain antibody directed to a cell surface tumor antigen. A non-limiting working example provided herein involves the use of a HER-2 affibody that binds HER-2 on the tumor surface coupled with PL. In other embodiments the targeting element is a ligand for a cell surface receptor on a tumor cell. A non-limiting working example provided herein involves the use of EGF that binds to EGFR on the tumor surface coupled with PL.

There is an increasing attention in recent years on exploring neoantigens for cancer immunotherapy. Unlike tumor associated tumor antigens, neoantigens are derived from nonsynonymous mutations in tumor cell genome and are thus strictly tumor-specific. However, one of the challenges facing neoantigen-based immunotherapy is that the neoepitopes are usually not shared among cancer patients. The current approach of first identifying these neoantigens by exome sequencing followed by synthesizing and delivering the antigenic epitopes to each individual patient is cumbersome and can only be applied to cancer patients on a case-by-case basis. In principle, oncolytic virotherapy would offer a simple means to release these neoantigens in individual patients, ensuring their efficient and timely presentation to the host's immune system. However, such a capability has not heretofore been examined. The examination on FusOn-Affibody-PL treated CT-26 tumor cells revealed that the armed virus has the capability in inducing neoantigen-specific antitumor immunity for neoantigenic peptides for both MHC Class I and Class II. The surprising data presented herein represents the first demonstration that virotherapy can induce measurable neoantigen-specific antitumor immunity.

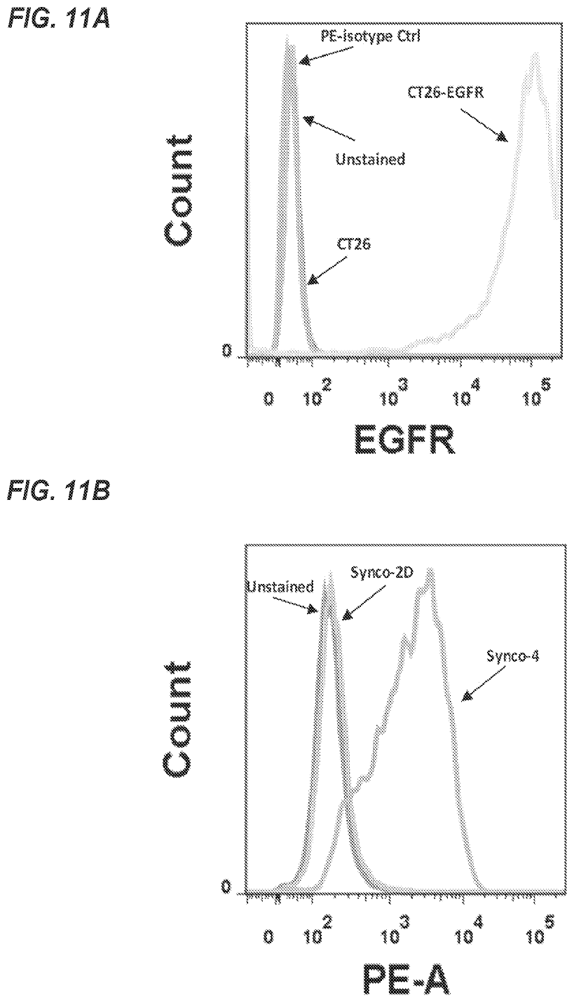

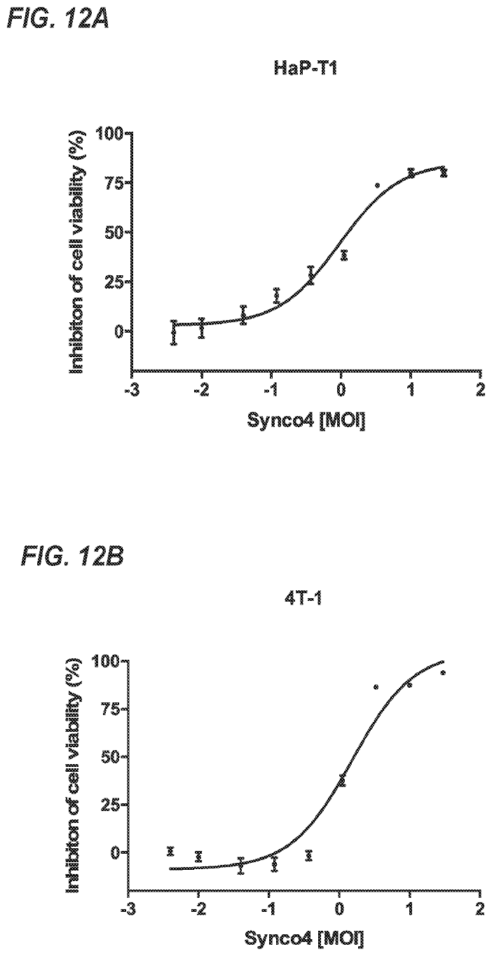

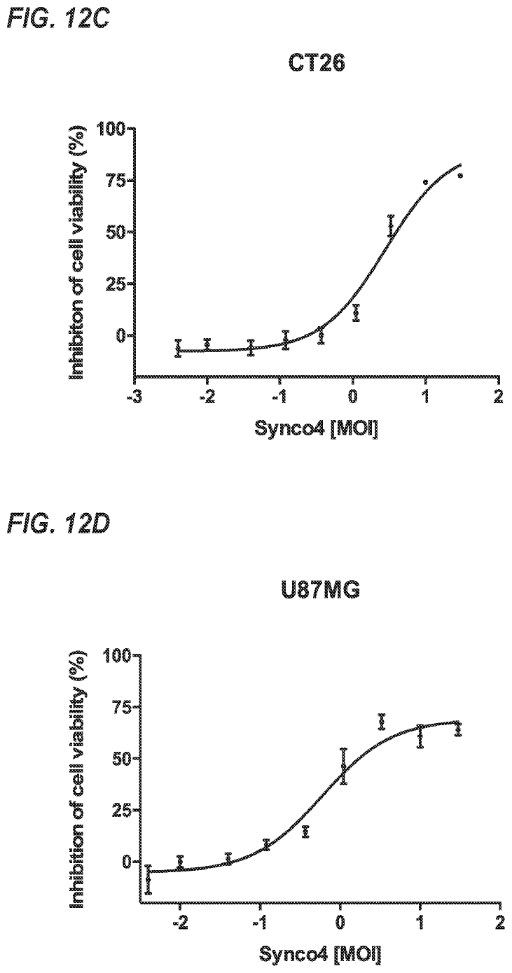

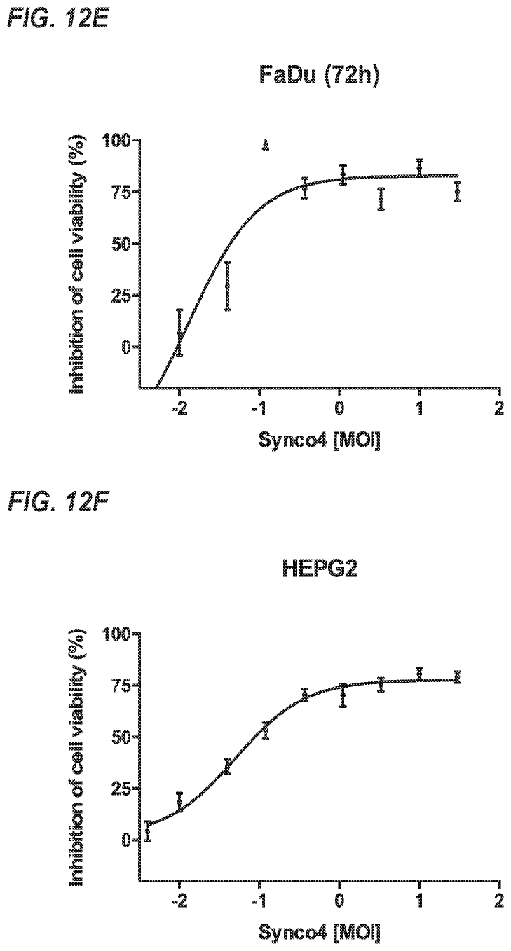

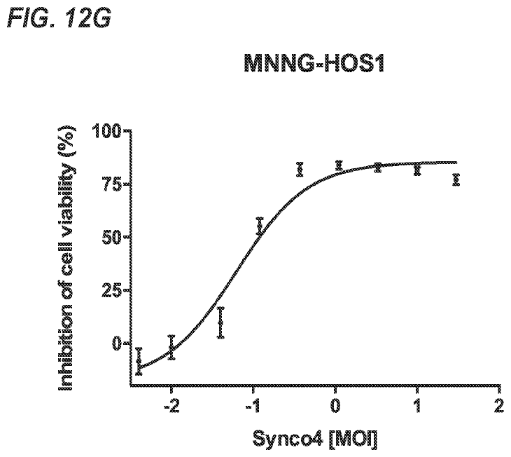

Although the design of Affibody-PL limits its application to tumor cells overexpressing HER2, this same strategy can be readily adapted to target other tumors by replacing Affibody with other binding moiety such as a ligand for different growth factor receptors or a single chain antibody. Indeed, as provided herein, in one embodiment the Affibody in the Affibody-PL chimeric construct was replaced with the binding domain of epidermal growth factor (EGF) to its receptor, and it was found that this construct can efficiently target tumor cells that overexpress EGFR (FIG. 11A). Additionally, it was also found that either of EGF-PL (or Affibody-PL for the same matter) can be incorporated into a HSV-1-based oncolytic virus to produce a similar potentiation effect (FIG. 12). Thus, it is anticipated that the disclosed strategy can be widely adapted, including the possibility to oncolytic viruses derived from other viruses, for treating a variety of malignant diseases.

The following examples are included for the sake of completeness of disclosure and to illustrate the methods of making the compositions and composites of the present invention as well as to present certain characteristics of the compositions. In no way are these examples intended to limit the scope or teaching of this disclosure.

### Example 1

### Constructions of the Disclosed Embodiments

FIG. 1A and FIG. 1B depict the design of an exemplary Affibody-PL and its in vivo action mechanism in tumor microenvironment. Affibody molecules are short peptides of 58 amino acid long, and are based a three-alpha-helical Z-domain scaffold that can be selected from combinatorial libraries to bind to a particular protein target with strong affinity and specificity. See Feldwisch J, et al. “Engineering of affibody molecules for therapy and diagnostics” Methods Mol Biol. 899 (2012) 103-26. Examples of suitable affibodies selected for strong binding affinity to HER2 are available. See Orlova A, et al. “Tumor imaging using a picomolar affinity HER2 binding affibody molecule” Cancer Res. 66(8) (2006) 4339-48; Steffen A C, et al. “Affibody-mediated tumour targeting of HER-2 expressing xenografts in mice” Eur J Nucl Med Mol Imaging 33(6) (2006) 631-8. The coding sequence from the five immunoglobulin-binding domains (B1-B5) was chosen from Protein L of Peptostreptococcus magnus as reported by Kastern et. al. (supra). Anti-HER2 Affibody and PL sequences were fused together in frame for the construction of Affibody-PL. A signal peptide (Sp) was added to the N-terminus to enable the chimeric molecule as a soluble form. FIG. 1A depicts the gene cassette of the chimeric molecule Affibody-PL that can guide innate immune cells to attack tumor cells through a serious of intermolecular engagements. On the sequential order from left to right: Rous sarcoma virus (RSV) long terminal repeat (LTR) (SEQ ID NO:4), a synthetic signal peptide (Sp) (SEQ ID NO:5) (as reported by Barash et al, Human secretory signal peptide description by hidden Markov model and generation of a strong artificial signal peptide for secreted protein expression. Biochemical and Biophysical Research Communications 294 (2002) 835-842), anti-HER2 Affibody (Affibody) (SEQ ID NO:3), Linker-HA tag (HA) (SEQ ID NO:6), the B1-B5 immunoglobulin binding domains of Protein L (SEQ ID NO:2), and bovine growth hormone polyadenylation signal (polyA) (SEQ ID NO:7). As one of skill in the art appreciates, other polyadenylation sites such as for example the SV40 PolyA (SEQ ID NO: 28) could be alternatively used. The actual length of the coding sequence of each component is not proportional to the size of the drawn box.

The schematic diagram in FIG. 1B illustrates an action mechanism of Affibody-PL once it is delivered to the tumor tissues by an oncolytic virus. Local administration of virotherapy will bring all the components together in the tumor microenvironment. The linchpin to trigger the illustrated chain intermolecular reaction is the soluble form of Affibody-PL, which can simultaneously bind to HER2-expressing tumor cells through Affibody and immunoglobulins (Igs) through PL. The Fc region of the Igs can subsequently bind and crosslink the Fc receptors on the surface of NK cells and macrophages, resulting in the activation of these innate immune cells and the killing of tumor cells. This strategy is envisaged to potentiate the overall antitumor effect of an oncolytic virotherapy on two fronts. First, it produces additional bystander antitumor activity by engaging innate immune cells with tumor cells. Second, it diverts two of the major components of antiviral immunity away from clearing the introduced oncolytic virus—the neutralizing antibodies and the innate immune cells including NK cells and macrophages, allowing virotherapy to exhibit maximal oncolytic effects. In the predicted action mechanism of Affibody-PL after delivered to HER2-expressing tumors by an oncolytic virus depicted in FIG. 1B, each of the key components is labeled. The administered oncolytic virus will express Affibody-PL in situ as well as attract innate immune cells such as NK cells and macrophages (Mc)) to the tumor site. The secreted Affibody-PL engages the infiltrated innate immune cells with tumor cells through a series of intermolecular binding events: Affibody to HER2, PL to Igs and Igs (via Fc region) to NK cells or macrophages (via Fc receptor).

The composition of an embodiment of a chimeric virus is depicted in FIG. 2A. FIG. 2A specifically depicts the step-wise construction of FusOn-H2. Depicted on the top is the genome of wild type HSV-2 (w.t. HSV-2, wild-type HSV-2 strain 186). The HSV genome is about 152 kb long and it contains the terminal repeats (TRs) and internal repeats (IRs), which are marked accordingly, as are the ICP10 and ICP47 genes. HSV-2 possesses several unique features that have been previously exploited by the inventors to convert it into an oncolytic agent. The HSV-2 ICP10 gene, the counterpart of ICP6 in HSV-1, contains in its N-terminus a well-defined region (N-domain) that can bind and phosphorylate the GTPase-activating protein Ras-GAP, leading to activation of the Ras/MEK/MAPK mitogenic pathway and c-Fos induction and stabilization, which are prerequisites for efficient HSV-2 replication. Deletion of this domain from the viral genome impairs virus growth in normal cells, which usually have an inactive Ras signaling pathway. As the Ras signaling pathway is a key regulator of normal cell growth, it is aberrantly activated in most human tumors due to either mutations in the Ras genes themselves or alterations in upstream or downstream signaling components. The present inventors previously established that a mutant HSV-2 deleted for the N-domain of the ICP10 gene would be able to replicate selectively in these tumor cells and that its replication would be restricted in normal cells with an inactive Ras pathway. A mutant HSV-2 (FusOn-H2) was constructed by replacing the PK domain of the ICP10 gene with the DNA sequence encoding the enhanced green fluorescent protein (EGFP).

The construction and use of FusOn-H2 is described in U.S. Pat. No. 8,986,672, which is incorporated herein by reference in its entirety. For constructing FusOn-H2, the N-domain of the ICP10 gene is deleted and replaced with the EGFP gene under expression of the CMV (cytomegalovirus) immediate early promoter, leaving the C-terminal ribonucleotide reductase (RR) domain intact and resulting in a modified ICP10 (mICP10, SEQ ID NO:8). Briefly, the HSV genome region containing the ICP10 left-flanking region (equivalent to nucleotide span 85994-86999 in the HSV-2 genome) was amplified with primer oligos 5′-TTGGTCTTCACCTACCGACA (SEQ ID NO:11), 3′-GACGCGATGAACGGAAAC (SEQ ID NO:12), while the RR domain and the right-flank region (equivalent to nucleotide span 88228-89347) were amplified with primer oligos 5′-ACACGCCCTATCATCTGAGG (SEQ ID NO:13), 3′-AACATGATGAAGGGGCTTCC (SEQ ID NO:14). In other words, this equates to a deletion of nucleotides 87000-87023, which is the ICP10 promoter region, and a deletion of nucleotides 87024-88226, which is the first 1,204 nucleotides of ICP10 domain (i.e. a deletion of amino acids 1 to 402 of the endogenous ICP10 polypeptide). The two PCR products were cloned into pNeb193 through EcoRI-NotI-XbaI ligation, generating pNeb-ICP10-deltaPK. Then the DNA sequence containing the CMV promoter-EGFP gene was PCR amplified from pSZ-EGFP with primer oligos 5′-ATGGTGAGCAAGGGCGAG (SEQ ID NO:15), 3′-CTTGTACAGCTCGTCCATGC (SEQ ID NO:16). The PCR-amplified DNA was then cloned into the deleted PK locus of pNeb-ICP10-deltaPK through BglII and NotI ligation, generating pNeb-PKF-2. During the design of PCR amplification strategies, the EGFP gene was fused in-frame with the remaining RR domain of the ICP10 gene, so that the new protein product of this gene contained the intact EGFP, which would facilitate the selection of the recombinant virus in the following experimental steps. The modified ICP10 gene was inserted into the virus by homologous recombination in which the pNeb-PKF-2 plasmid DNA was cotransfected with purified wt186 virion DNA into Vero cells by use of Lipofectamine (Invitrogen, Carlsbad, CA, USA). The recombinant virus was screened and identified by selecting GFP-positive virus plaques. During screening, all the GFP positive plaques showed clear formation of syncytia among the infected cells, indicating that this modified virus might induce widespread cell membrane fusion. A total of six plaques were picked of which FusOn-H2 was selected for further characterization.

FIG. 2B depicts the step-wise construction of an embodiment of a FusOn-Affibody-PL, which was constructed by initially deleting the EGFP gene from FusOn-H2, and then inserting the Affibody-PL gene cassette (SEQ ID NO:9) in the region next to the modified ICP10 gene on the HSV-2 genome.

### Example 2

### Affibody-PL Engagement of Innate Immune Cells

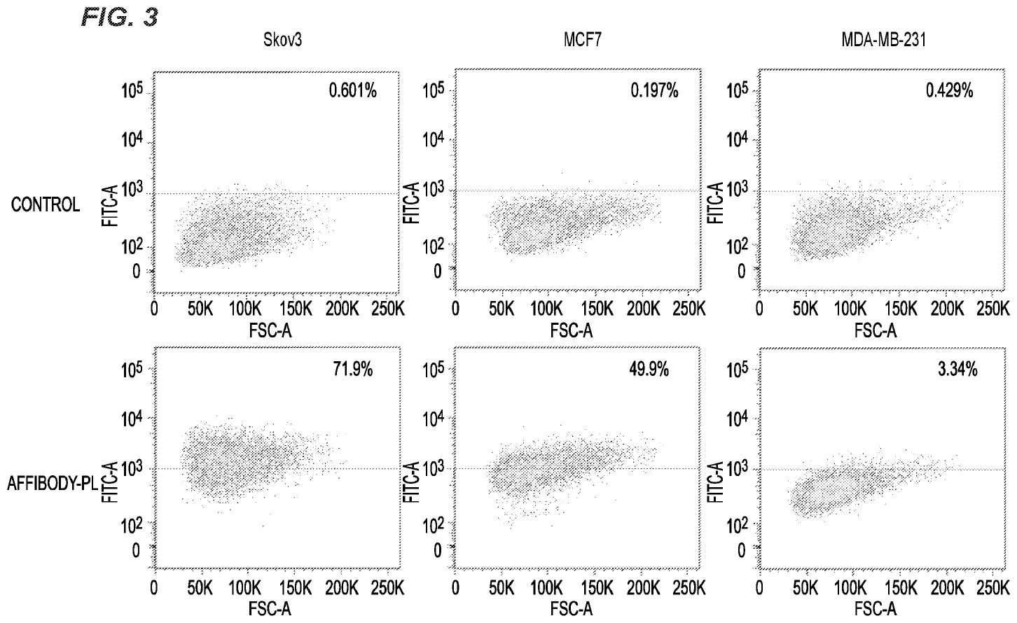

Efforts were undertaken to demonstrate that Affibody-PL can actively engage innate immune cells to attack tumor cells when tested in vitro. First, the ability of Affibody-PL to selectively bind to tumor cells expressing HER2 was examined. A plasmid containing the gene cassette, pcDNA-Affibody-PL that was constructed by inserting the Affibody-PL into the pcDNA3 plasmid, or a control plasmid (pcDNA3-EGFP from Addgene) was transfected into 293 cells and supernatants were collected 24 hours (hr) later. The supernatants (100 ul) were added to three tumor cell lines (Skov3, derived from a serous cystoadenocarcinoma; MCF7; and MDA-MB-231) that express varying levels of HER2 to let Affibody-PL first bind to HER2 on tumor cell surface. After washing, FITC-conjugated anti-HA-tag antibody was added. The stained cells were then analyzed by flow cytometry and the results were shown in FIG. 3. As shown, Affibody-PL binds efficiently to Skov3 cells that express high level of HER2; more than 70% of cells stained positive. For MCF7 cells that express moderate levels of HER2, approximately half of the cells were positively stained. For the triple negative MDA-MB-231 breast cancer cells, only 3% of cells were positively stained. These results show that Affibody in the chimeric construct allows the molecule to bind strongly to HER2-expressing tumor cells.

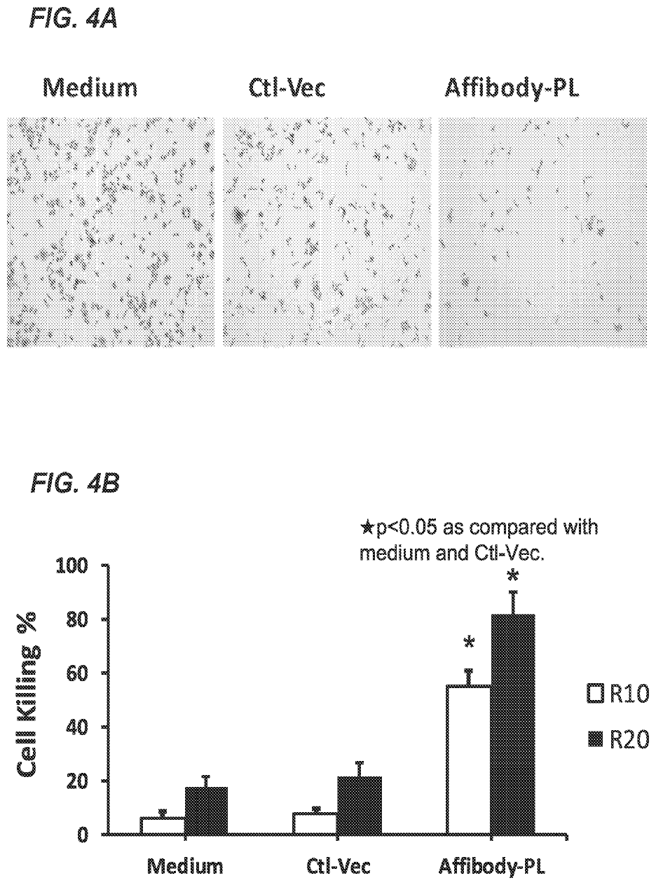

Next, another in vitro experiment was conducted to test if Affibody-PL can initiate the anticipated intermolecular interactions shown in FIG. 1B to guide the innate immune cells to kill HER2-expressing tumor cells. To more closely mimic the actual in vivo situation, peripheral blood mononuclear cells, which contain NK cells and monocytes at the range of 5-20% and 10-30%, respectively were chosen as the source of innate immune cells. SKOV3 cells were mixed with PBMCs in the presence of immunoglobulins (Igs, from Sigma) and Affibody-PL-containing supernatant or the control supernatant. Twenty hours later, PBMCs were washed away, and the viability of tumor cells was initially examined by direct visualization under microscope after they were stained with 0.1% crystal violet-ethanol solution (FIG. 4A). The killing effect on tumor cells was further quantitated by lysing the cells in 2% SDS and the released dye was measured at 595 nM wavelength using Spectramax 5 plate reader (FIG. 4B). The results show that the presence of Affibody-PL led to a significant killing of tumor cells when compared to the mixture without this chimeric molecule. This effect was particularly obvious when the effector to target (E:T) ratio was relatively low (10:1) and the wells with the control supernatant show very little tumor cell killing while nearly 70% of cell killing was detected in the wells with Affibody-PL. With the E:T ratio at 20 to 1, there was a notable increase on the background killing in both wells with the medium alone and the control supernatant. However, the tumor cell killing in the wells with Affibody-PL was further increased to over 90 percent. Together, these results demonstrate the capability of Affibody-PL in guiding innate immune cells to kill tumor cells through a series of intermolecular engagements.

### Example 3

### Insertion of Affibody-PL Coding Sequence into an Oncolytic HSV and In Vitro Characterization of the Armed Virus

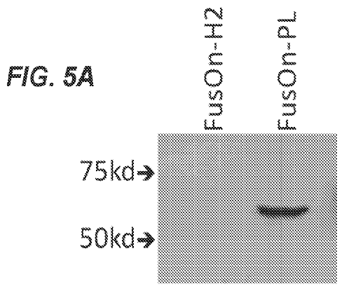

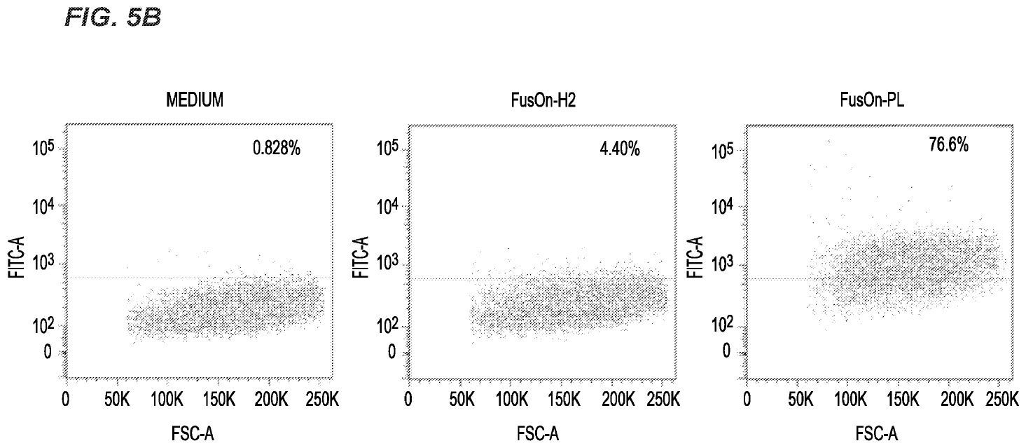

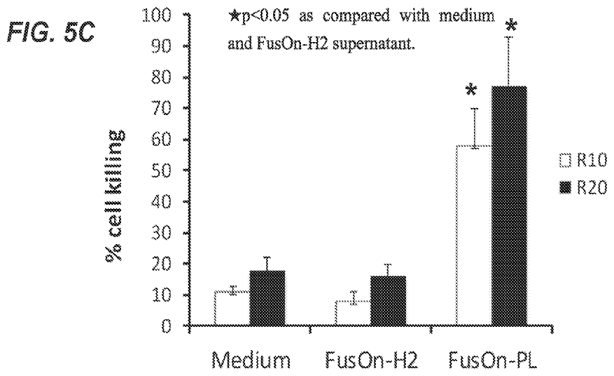

Next, the Affibody-PL coding sequence was inserted into the genome of an HSV-2-based oncolytic virus, FusOn-H2, through the homologous recombination as illustrated in FIG. 2A and FIG. 2B by a technique previously described in publications of certain of the inventors. See Fu, X., et al. FusOn-H2 is a mutant type 2 herpes simplex virus deleted for the protein kinase domain of the ICP10 gene and is a potent oncolytic virus (Mol Ther. 13(5) (2006) 882-90; Fu X, et al. “Construction of an oncolytic herpes simplex virus that precisely targets hepatocellular carcinoma cells” Mol Ther. 20(2) (2012) 339-46); each of which are incorporated herein by reference. The entire sequence of the derived FusOn-Affibody-PL is provided in SEQ ID NO: 17. The expression of Affibody-PL from the new virus, FusOn-PL, was confirmed by Western blot analysis of the supernatant collected from cells infected with the virus, which shows that Affibody-PL is abundantly expressed by FusOn-PL (FIG. 5A). The property of the Affibody-PL expressed from FusOn-PL was assessed by measuring its ability to bind to HER2-expressing tumor cells and its ability to guide innate immune cells to kill tumor cells in the same way as described in FIG. 3 and FIG. 4A-FIG. 4B, respectively, but using cells of murine origin instead. For tumor cells, the CT26 murine colon cancer cell line that had been stably transduced with HER2 was used. See Penichet M L, et al. “In vivo properties of three human HER2/neu-expressing murine cell lines in immunocompetent mice” Lab Anim Sci. 49(2) (1999) 179-88. For the effector cells, splenocytes harvested from immune competent Balb/c mice were used. Flow cytometry analysis on the binding of Affibody-PL produced from FusOn-PL to murine tumor cells expressing HER2 is shown in FIG. 5B. The experiment was similarly conducted as in FIG. 3, with the exception that a murine colon cancer cell line that was stably transduced with HER2 (CT26-HER2) was used. The results in FIG. 5C in which tumor cells were mixed with PBMCs, showed that, similar to those in FIG. 4B, a significant killing of tumor cells was observed in the well where supernatant containing Affibody-PL was added when compared to the other two wells without this ingredient. Together, these results demonstrate that Affibody-PL could also efficiently guide innate immune cells of murine origin to kill murine tumor cells if they express the targeted tumor antigen.

### Example 4

### Therapeutic Evaluation of FusOn-PL

To evaluate the therapeutic effect of FusOn-PL and to compare it with that of the parental FusOn-H2, the murine CT26 colon tumor model that is only marginally sensitive to the therapeutic effect of FusOn-H2 was chosen. This would allow the therapeutic benefit from the incorporated Affibody-PL to be fully evaluated. Specifically, a CT26 cell line that was stably transduced with the HER2 gene was used for this experiment. CT26-HER2 tumor cells were implanted subcutaneously to the right flank of syngeneic immune-competent BALB/c mice. Once tumors reached the approximate size of 5 mm in diameter, mice were treated intratumorally with the same dose of either FusOn-PL or FusOn-H2. Two mice from each group were euthanized at day 3 after treatment to collect tumor tissues for measurement of NK cell infiltration, and the remaining animals were kept for 4 weeks to evaluate the therapeutic effect by monitoring tumor size after treatment.

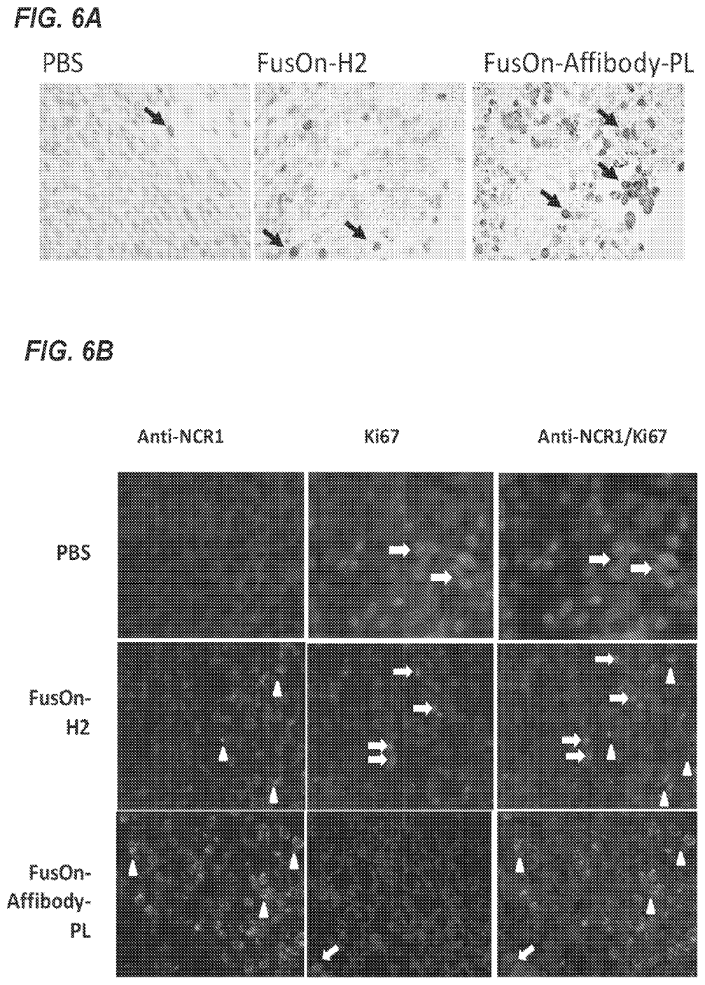

Immunohistochemical staining on the collected tumor tissues show that NK cells were only scarcely detected in the PBS-treated control tumors, a finding which is consistent with the reports in the literature that NK cells were mostly detected at the frequency of 1-3 per microscopic intratumoral field. As shown in FIG. 6A, NK cells were readily detectable in tumors treated with both FusOn-H2 and FusOn-PL, consistent with the reports that HSV-2 infection can trigger significant infiltration of NK cells to the infection site. Surprisingly, the presence of NK cells in tumor tissues treated with FusOn-PL was particularly profound and significantly more than those from FusOn-H2 treatment.

To determine if the increased presence of NK cells in FusOn-PL treated tumors was due to an enhanced local NK cell proliferation, the tumor tissues were doubly stained for both NK cell marker (NCR1) and Ki67 protein. The results presented in FIG. 6B showed that, although tumor cells were strongly stained with ki67 (particularly in the PBS treated tumor tissue), there was no significant Ki67 staining on NK cells in tumor tissues treated either with FusOn-PL or FusOn-H2. The results thus exclude the possibility of Affibody-PL in stimulating NK cell proliferation and suggest that the increased NK cell presence during FusOn-PL treatment is due to a positive feedback loop on their recruitment. Regardless, the increased presence of NK cells in FusOn-PL treated tumor tissues would allow the secreted Affibody-PL to act effectively to guide them to kill tumor cells

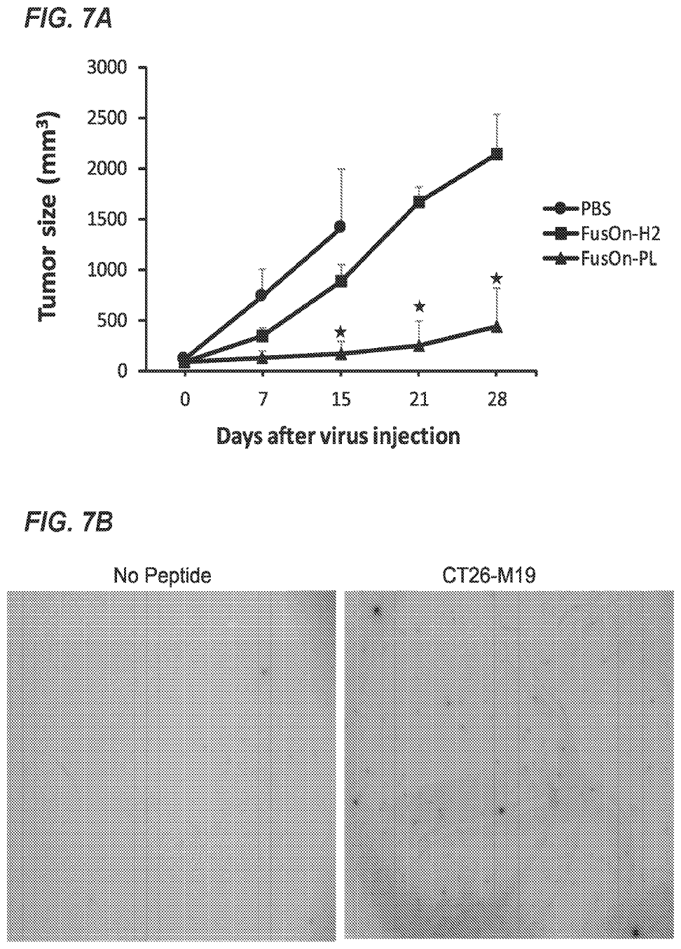

FIG. 7A showed the results from an in vivo experiment that was designed to directly compare the therapeutic efficacy of FusOn-PL with the parental FusOn-H2 by treating mice bearing tumors that were HER2 positive. The results show that administration of FusOn-H2 only slightly slowed down the tumor growth when compared with the PBS control. In contrast, FusOn-PL treatment effectively prevented tumor growth for an extended period of time. The treated tumors were significantly smaller than those in the FusOn-H2 treated group. By the end of the experiment, five out of the ten mice in FusOn-PL treated group were completed tumor free, while no tumor-free animals were detected in other treatment groups. Together, these results demonstrate that incorporation of Affibody-PL into an oncolytic HSV can enhance the antitumor effect of the virotherapy, and this potentiation strategy may be particularly beneficial to the patients in which the tumors are relatively resistant to the direct oncolytic effect of the applied virus.

The tumor-free mice in FusOn-PL treated group were subsequently challenged with fresh CT26-HER2 tumor cells implanted at the left flank. All three mice were completely protected and no trace of tumor was detected from the challenge for more than 4 weeks. The tumor challenge was not done to mice in the other two treatment groups, as all the mice had to be euthanized due to the large tumor burden. The challenged mice were kept for 4 more weeks to monitor tumor growth. No tumor formation was detected in any of the mice by the end of the experiment, indicating a robust antitumor immunity might have been generated from FusOn-PL treatment that subsequently provided the complete protection of these mice from tumor challenge.

Tumor cells contain frequent point mutations that can result in neoantigen formation. Inducing immune responses to these neoantigens is particularly appealing for cancer immunotherapy, as theoretically they are strictly tumor-specific. The neoantigen profile of CT-26 cells has recently been reported by Kreiter et al. See Kreiter S, et al. “Mutant MHC class II epitopes drive therapeutic immune responses to cancer” Nature 520 (7549) (2015) 692-6. To determine if FusOn-PL-mediated tumor cell killing could induce anti-neoantigen immunity, 3 mutated peptides (as listed in TABLE 1) that predictably contain MHC Class I neoantigen epitopes were initially chosen to examine if any cytotoxic T cells specific for these antigens could be detected in these protected mice. The splenocytes collected from the mice were stimulated with these peptides or a control peptide. The specificity of T cell response was determined by ELISPOT assay. The results show that splenocytes from one mouse reacted to a single peptide from this assay (FIG. 7B), indicating that induction of T cell response to these Class I neoantigen epitopes during FusOn-PL virotherapy could be detectable.

TABLE 1

| | | | | T | MHC |
|---|---|---|---|---|---|
| | | Mutated sequence | Muta- | cell | I |
| Name | Gene | synthesized | tion | type | score |
| CT26- | Tmem87a | GAIIVRGCSMPGPWRSGRL | G63R | CD8⁺ | 0.7 |
| M19 | | LVSRRWSVE | | | |
| | | SEQ ID NO: 18 | | | |
| CT26- | Als2 | GYISRVTAGKDSYIALVDK | L675I | CD8⁺ | 0.2 |
| M39 | | NIMGYIAS | | | |
| | | SEQ ID NO: 19 | | | |
| CT26- | E2F8 | VILPZAPSGPSYATYLQPA | I522T | CD8⁺ | 0.1 |
| M26 | | QAQMLTPP | | | |
| | | SEQ ID NO: 20 | | | |

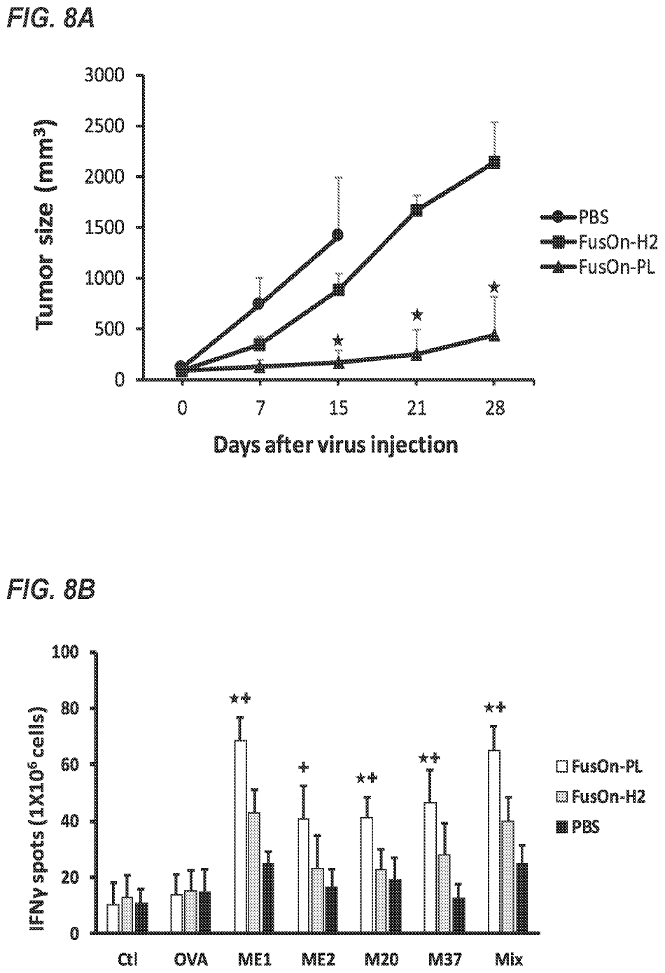

To further characterize the neoantigen-specific immune response during FusOn-PL and FusOn-H2 virotherapy, the in vivo animal experiment shown in FIG. 6A and FIG. 6B was essentially repeated. The only difference was that all animals were euthanized at day 21 after virotherapy, when all animals were still alive, and the spleens from the animals were collected for further analysis of anti-neoantigen immunity. The therapeutic data collected from this second in vivo experiment showed that, even with this relatively short treatment period, FusOn-PL was still significantly more effective than FusOn-H2 at preventing the tumor growth at day 21 after the start of virotherapy (FIG. 8A), with approximately 40% of mice showing tumor-free by the end of the experiment.

The studies by Kreiter et al (supra) in three independent murine tumor models including CT-26 showed that the majority of the immunogenic mutanome was recognized by CD4+ T cells. To further characterize neoantigen-specific immunity, we chose a panel of MHC class II neoantigen peptides from the report by Kreiter et al. (listed in TABLE 2) to measure the antitumor immunity in these animals.

TABLE 2

| | | | | T | MHC |
|---|---|---|---|---|---|
| | | Mutated sequence | Muta- | cell | II |
| Name | Gene | synthesized | tion | type | score |
| CT26- | Aldh18a1 | LHSGQNHLKEMAISVLEARA | P154S | CD4⁺ | 0.05 |
| ME1 | | CAAAGQS | | | |
| | | SEQ ID NO: 21 | | | |
| CT26- | Ubqln1 | DTLSAMSNPRAMQVLLQIQQ | A62V | CD4⁺ | 0.24 |
| ME2 | | GLQTLAT | | | |
| | | SEQ ID NO: 22 | | | |
| CT26- | Slc4a3 | PLLPFYPPDEALEIGLELNSS | T373I | CD4⁺ | 0.9 |
| M20 | | ALPPTE | | | |
| | | SEQ ID NO: 23 | | | |
| CT26- | DHX35 | EVIQTSKYYMRDVIAIESAW | T646I | CD4⁺ | 0.1 |
| M37 | | LLELAPH | | | |
| | | SEQ ID NO: 24 | | | |

FIG. 8B shows the enumeration data of ELISPOT assay on these neoantigen peptides and the mixture of them. The controls include no peptide or an unrelated peptide (the ovalbumin Class II peptide (OVA 323-339). The results show that significant increase on ELISPOT staining was detected in the animals treated with FusOn-PL on all four Class II neoantigen peptides, although the magnitude of the response varies among the individual neoantigens (FIG. 8B). Taken together, these data demonstrate that tumor destruction by FusOn-PL can induce neoantigen-specific antitumor immunity on both Class I and Class II MHC epitopes, while the parental FusOn-H2 is less efficient in inducing such an immunity.

### Example 5

### HSV-1 Constructs Expressing PL

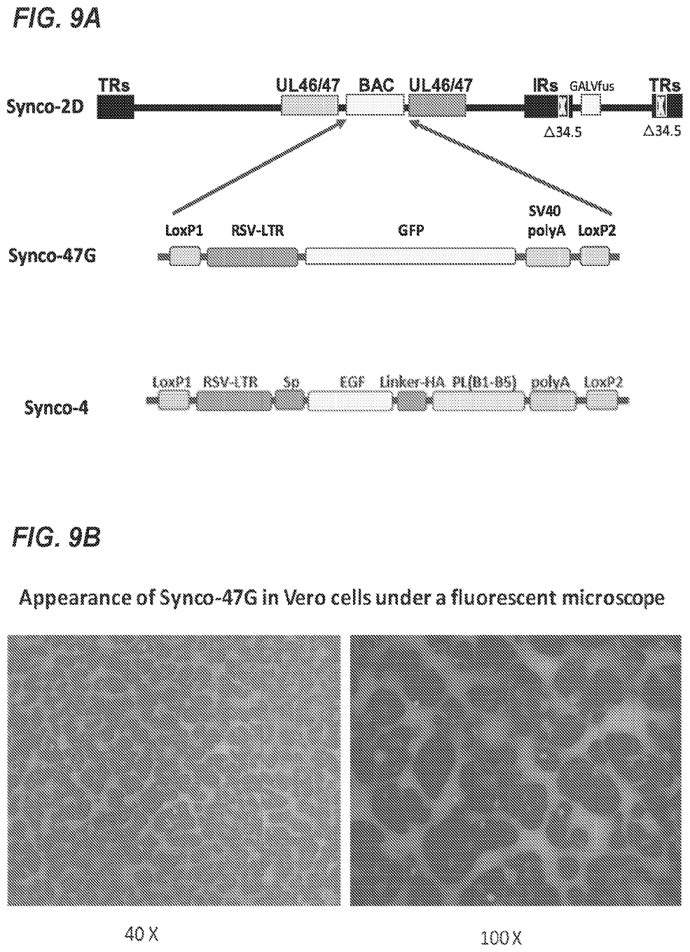

PL expressing constructs of HSV-1 oncolytic viruses was also accomplished where the tumor cell binding site was designed to be the epidermal growth factor receptor (EGFR). Alternative embodiments have an Affibody to a tumor cell antigen, such as but not limited to HER2 may be included in lieu of the EGFR. FIG. 9A shows a schematic of Synco-4 construction beginning with Synco-2D. As detailed in prior studies by the inventors and incorporated herein by reference, Synco-2D, is a HSV-1-based oncolytic virus, which was constructed by an intended process that can be summarized as: 1) deletion of both copies of the ICP34.5 gene (indicated as Δ34.5), 2) by insertion of the hyperfusogenic glycoprotein from gibbon ape leukemia virus (GALV.fus) into the Us3 gene (ultimately resulting in inactivation of this gene), and 3) insertion the bacterial artificial chromosome (BAC) into the intergenic region of UL46 and UL47. This virus can induce potent cell membrane fusion due to the intrinsic fusogenic phenotype as well as the insertion of GALV.fus.

More specifically, Synco-2D was constructed by a process starting with fHSV-Δ-pac, a BAC-based construct that contains a mutated HSV genome, in which the diploid gene encoding γ34.5 and both copies of HSV packaging signal were deleted. See Saeki, Y., et al. “Herpes simplex virus type 1 DNA amplified as bacterial artificial chromosome in Escherichia coli: rescue of replication-competent virus progeny and packaging of amplicon vectors. Hum. Gene Ther. 9 (1998) 2787-2794, 1998. Infectious HSV cannot be generated from this construct unless an intact HSV packaging signal is provided in cis; otherwise, the virus will be replication conditional due to the deletion of both copies of γ34.5. Thus the construct “Baco-1” was constructed by inserting a DNA sequence containing a HSV packaging signal and an enhanced GFP gene cassette into the unique Pad restriction site located in the BAC sequence in fHSV-Δ-pac, as described previously. See Fu et al. “Expression of a Fusogenic Membrane Glycoprotein by an Oncolytic Herpes Simplex Virus Potentiates the Viral Antitumor Effect, Molec Ther 7 (6) (2003) 748-754. To generate Synco-2D, Baco-1 was initially subjected to random mutagenesis. The syncytial phenotype was identified by screening the mutagenized virus on Vero cells. The circular form of viral DNA was then obtained from the new virus (Baco-F1) by extracting virion DNA from Vero cells shortly (1 h) after virus infection. The viral DNA was then transformed into the competent E. cell DH-10B through electroporation, and Baco-F1 DNA was purified from bacterial growth with the use of a Qiagen kit. To insert the hyperfusogenic glycoprotein gene of gibbon ape leukemia virus (GALV.fus) into Baco-F1, the gene cassette encoding GFP in Baco-F1 was replaced with GALV.fus (driven by the conditional UL38 promoter of HSV) through an enforced ligation strategy. The ligation mixture was directly transfected into Vero cells using LipofectAMINE (Life Technologies, Inc.) and incubated for 3-5 days to permit the generation of infectious virus. The resultant viruses were subsequently plaque-purified. See Nakamori, M., et al., “Effective Therapy of metastatic ovarian cancer with an oncolytic herpes simplex virus incorporating two membrane-fusion mechanisms” Clinical Cancer Res. 9(7) (2003) 2727-2733, which is incorporated herein by reference in its entirety. Subsequent characterization of Synco-2D led to the surprising finding that, despite this design, the GALV.fus gene cassette was inserted into the Us3 gene through unpredicted recombination.

For Synco-4 construction, a gene cassette was initially inserted into Synco-2D to replace the BAC sequence contained in the viral genome by homologous recombination. This enabled the new virus, Synco-47G, to be easily identified by the green color under microscope (as shown in FIG. 9B). A gene cassette that contains EGF-PL fusion gene (EGF ligand, SEQ ID NO:10) connected for in-frame expression to a Protein L (Protein L construct of SEQ ID NO:2) driven by the RSV-LTR and flanked by two LoxP sites (SEQ ID NO: 26 and SEQ ID NO: 27) was inserted into Synco-47G to replace the GFP gene, so that Synco-4 could be selected as a “white” virus from the “green” background.

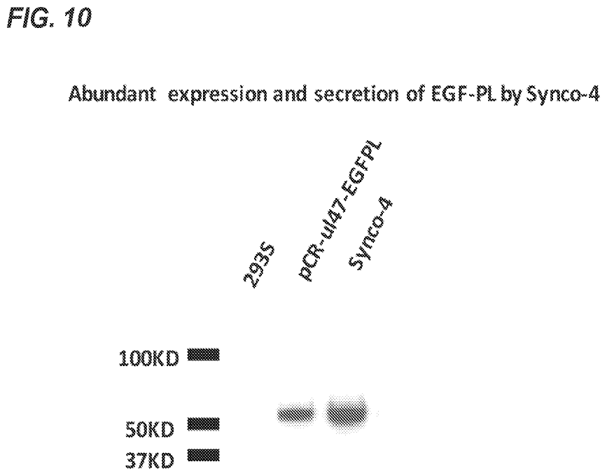

As shown in FIG. 10, abundant expression and secretion of EGF-PL by Synco-4 was observed in 293 cells that were either transfected with a plasmid (pCR-ul47-EGFPL) that contains the EGF-PL gene cassette (as shown in FIG. 9A) or infected with Synco-4. 293 cells without either transfection or infection were used as a negative control. Forty-eight hrs after transfection/infection, supernatant was collected and the secreted EGF-PL was detected by Western blot analysis. The results show that there is an abundant presence of EGF-PL in the supernatant of 293 cells infected with Synco-4, indicating that the transgene is efficiently expressed by the virus.

As shown in FIG. 11A and FIG. 11B, EGF-PL produced by Synco-4 binds to EGFR expressed on tumor cells with high efficiency and specificity. The experiment in FIG. 11A was conducted by incubating the same supernatant harvested from pCR-ul47-EGFPL-transfected cells as mentioned above in with either CT26 or CT26-EGFR, followed by reaction with PE-conjugated Ig. The result shows flow cytometry of the staining for EGFR expression on CT-26-EGFR cells but not on other tumor cells. The experiment in FIG. 11B was conducted by incubating the same supernatant harvested from Synco4-infected cells as mentioned above in with either CT26 or CT26-EGFR, followed by reaction with PE-conjugated Ig. The result shows flow cytometry evidencing that EGF-PL in the supernatant of Synco-4 infection can strongly bind to CT26-EGFR cells, and the supernatant from the parental FusOn-H2 did not show any binding.

As shown in FIGS. 12A-G, Synco-4 can induce dose-dependent inhibition of tumor cell proliferation on Syrian Hamster adenocarcinoma cells (HaP-T1) (FIG. 12A), Murine breast cancer cells (4T-1) (FIG. 12B), Murine colon cancer cells (CT-26) (FIG. 12C), Human glioblastoma cells (U87) (FIG. 12D), Human pharynx squamous carcinoma cells (FaDu) (FIG. 12E), Human hepatocellular carcinoma cell (HEPG2) (FIG. 12F), and Human osteosarcoma cells (MNNG-HOS1) (FIG. 12G). HaP-T1 cell was obtained from European Collection Authenticated Cell Culture (ECACC). Other cell lines, 4T-1 cell, CT-26 cell, U87 cell, FaDu cell, HEPG2 cell, MNNG-HOS1 cell, were purchased from ATCC. For the experiment, 5000 cells/well were placed in 96-well plate and cultured overnight, culture media were removed and treated with 100 μl serum-free media containing various concentrations of Synco-4 from 0.003-3 multiplicity of infection (MOD. After 2 hr infection, media were removed and replaced with 100 μl culture media for continuous incubation for additional 48 hrs. Cells were detected with MTT assay kit (BioFloxx, Germany) followed manufacture's instruction. Briefly, 5 μl MTT solution (5 mg/ml) were added and incubated for 4 hrs in dark, then the media were removed and 150 μl DMSO was added. Then, the mixture was incubated at RT for 5 min with shaking, and the absorbance value (OD) thereof was determined at wavelength 490 nm. Cell viability was calculated by normalizing vehicle treatment and presented as inhibition rate. All experiments were repeated 3 times, n=3-6.

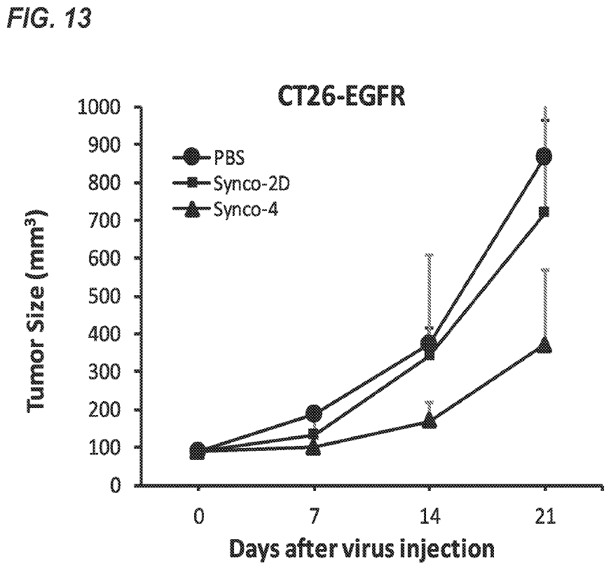

As shown in FIG. 13, Synco-4 has an enhanced antitumor activity over the parental Synco-2D against CT26-EGFR tumor. CT26-EGFR tumor cells were implanted subcutaneously to the right flank of Balb/c mice. Once tumors reached the approximate size of 5 mm size in diameter, mice were divided into 3 groups and treated with: 1) PBS at the same volume (100 μl), 2) 2×10⁷ pfu of Synco-2D, and 3) 2×10⁷ pfu of Synco-4. Tumor size was measured periodically and plotted. The results show that Synco-4 has an enhanced antitumor activity as compared with Synco-2D against CT26-EGFR tumor.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. An improved oncolytic virus comprising an oncolytic virus backbone genetically modified to encode and direct secretion from infected cells of a chimeric molecule comprising a tumor cell binding component and an immunoglobulin (Ig) aggregator component, wherein the secreted chimeric molecule increases bystander tumor cell killing and anti-tumor immunity in the presence of anti-viral antibodies or other Igs and innate immune cells.

2. The improved oncolytic virus of claim 1 wherein the immunoglobulin aggregator component includes at least one Ig-binding kappa light chain binding domain derived from a Peptostreptococcal Protein L.

3. The improved oncolytic virus of claim 2 wherein the immunoglobulin binding component includes at least four or five Ig-binding “B” domains derived from a Peptostreptococcal Protein L.

4. The improved oncolytic virus of claim 1 wherein the oncolytic virus backbone is based on Herpes Simplex Virus Type 1 (HSV1).

5. The improved oncolytic virus of claim 1 wherein the oncolytic virus backbone is based on Herpes Simplex Virus Type 2 (HSV2).

6. The improved oncolytic virus of claim 1 wherein the tumor cell binding component is either an affibody, or a ligand, or a short peptide, or a single chain antibody, or a single domain antibody, that binds to a tumor antigen.

7. The improved oncolytic virus of claim 6 wherein the tumor antigen is selected from human epidermal growth factor receptor 2 (HER2), epidermal growth factor receptor (EGFR), Erb-B2 Receptor Tyrosine Kinase 3 (ErbB3), epithelial cell adhesion molecule (EpCAM), mesothelin (MSLN), MET protooncogene, insulin-like growth factor 1 receptor (IGF1R), ephrin receptor A3 (EphA3), TNF receptor apoptosis-inducing ligand receptor 1 (TRAIL-R1), TNF receptor apoptosis-inducing ligand receptor 2 (TRAIL-R2), vascular endothelial growth factor receptor (VEGFR), receptor activator of nuclear factor κβ ligand (RANKL), programmed death-ligand 1 (PD-L1), phosphatase of regenerating liver 3 (PRL-3).

8. The improved oncolytic virus of claim 1 wherein the tumor cell binding component is an extracellular domain of epidermal growth factor (EGF) that binds to an EGF receptor (EGFR) on tumor cells.

9. The improved oncolytic virus of claim 4, wherein the HSV1 backbone comprises at least one deletion of ICP34.5.

10. The improved oncolytic virus of claim 5, wherein the HSV2 backbone comprises an N-domain deletion of an ICP10 enabling selective replication in tumor cells

11. An improved oncolytic virus with increased bystander cell killing and induced anti-tumor immunity, the virus comprising an oncolytic herpes virus backbone genetically modified to encode an Affibody-Protein L (PL) cassette comprising an Anti-HER2 Affibody and a plurality of Protein L immunoglobulin binding domains fused together in frame to form an Affibody-PL that is engineered for extracellular secretion by cells infected with the oncolytic virus.

12. The improved oncolytic virus of claim 12, wherein the Affibody-PL cassette comprises a synthetic signal peptide (Sp), the anti-HER2 Affibody (Affibody), a linker, the plurality of Protein L immunoglobulin binding domains, and a growth hormone polyadenylation signal (polyA).

13. An improved oncolytic virus with increased bystander cell killing and induced anti-tumor immunity, the virus comprising an oncolytic herpes virus backbone genetically modified to encode an extracellular domain of epidermal growth factor (EGF) that binds to an EGF receptor (EGFR) on tumor cells and a plurality of Protein L domains fused together in frame to form an EGF-PL that is engineered for extracellular secretion by cells infected with the oncolytic virus.

15. A method of treating cancer comprising administering a therapeutically effective amount of the improved oncolytic virus of claim 12 and a diluent or carrier.

16. The method of claim 15, wherein the improved oncolytic virus comprises SEQ ID NO:17.

15. A method of treating cancer comprising administering a therapeutically effective amount of the improved oncolytic virus of claim 14 and a diluent or carrier.

16. The method of claim 15, wherein the improved oncolytic virus comprises SEQ ID NO:25.

17. A fusion protein comprising a tumor cell binding component and an immunoglobulin (Ig) aggregator component, wherein the immunoglobulin aggregator component includes at least one Ig-binding kappa light chain binding domain derived from a Peptostreptococcal Protein L.

18. The fusion protein of claim 17, wherein the at least one Ig-binding kappa light chain binding domain derived from a Peptostreptococcal Protein L has at least 80% amino acid identity with SEQ ID NO:29.

19. The method of claim 15, wherein the improved oncolytic virus is administered to one or more of pancreatic cancer cells, breast cancer cells, colon cancer cells, glioma cancer cells, head and neck cancer cells, liver cancer cells, and bone cancer cells.
